(19) United States
(12) Patent Application Publication
(10) Pub. No.: US 2011/0144545 A1
(43) Pub. Date: Jun. 16, 2011

(54) Methods And System For Delivering Treatment To A Region Of Interest Using Ultrasound

(75) Inventors: Ying Fan, Niskayuna, NY (US); Cynthia Elizabeth Landberg Davis, Niskayuna, NY (US); Dhiraj Arora, Niskayuna, NY (US)

(73) Assignee: GENERAL ELECTRIC COMPANY, SCHENECTADY, NY (US)

(21) Appl. No.: 12/638,010

(22) Filed: Dec. 15, 2009

(51) Int. Cl. A61N 7/02

(52) U.S. Cl. 601/3

(57) ABSTRACT

A method for treating a region of interest using ultrasound is provided. The method comprises cavitating fat cells in the region of interest using one or more cavitating harmonics, and thermally treating connective tissues in the region of interest using one or more thermal harmonics.

### BACKGROUND

The invention relates to treatment of tissues, and more particularly to methods and systems for treating tissues using ultrasound.

Various body contouring systems exist today that attempt to remove or destroy subcutaneous fat tissue (or adipose tissue) from a person's body. Some systems are invasive, such as liposuction, where a device is inserted into the body and the device physically removes adipose tissue through suction. Other systems are non-invasive, for example, some laser based systems. Non-invasive systems are preferred due to relative comfort for the patient while receiving the therapy, and also there is usually no need for recovery time.

In some cases after a body contouring procedure is followed by one or more treatments to facilitate skin tightening or contour irregularity smoothing. The skin tightening is often carried out as procedures separate from the procedure for reducing adipose tissue. Skin tightening may be achieved by heating connective tissues in an area where adipose tissue reduction is carried out. Typically, adipose tissue reduction and skin tightening are performed using different equipment. For example, adipose tissue reduction may be performed by liposuction, whereas skin tightening may be achieved by radio frequency treatment.

High Intensity focused ultrasound (HIFU) can also destroy human body tissue. Typically in HIFU applications the ultrasound energy is focused so as to cause the tissue to be heated to a point of cellular death. However, if the energy of the ultrasound is applied in a pulsed mode or at lower frequencies the tissue is minimally heated and cells can be destroyed by a lysis effect. The cavitation effect becomes predominant when the acoustic power at the point of focus exceeds a determined threshold. This cavitation effect is linked to the formation of microscopic bubbles of gas that explode when they reach a critical diameter causing a local release of appreciable amounts of energy which leads to the destruction of neighboring tissue. The thermal effect is due to the acoustic absorption of the tissue, which converts the mechanical energy of the acoustic wave into thermal energy.

The temperature increase in the region of interest is directly linked to the acoustic power of the applied ultrasound field at the point of focus. In the case of a moderate temperature and a long duration of application, transfer and spreading of heat energy occurs around the point of focus, notably due to thermal conduction in the medium and to blood flow. This leads to poor control of the volume being treated, which may lead to healthy zones being destroyed.

In the case of elevated temperatures and a short duration of application, the acoustic power at the focal point exceeds the cavitation threshold and becomes destructive. This cavitation effect is particularly important at the various interfaces that the acoustic field encounters, for example at the skin, the muscles and the walls of organs. This leads to tissue destruction beyond the zone immediately around the focus of the transducer.

Due to the need for a different system to carry out the two procedures, the patient is required to visit the physician separately for adipose tissue reduction, and skin tightening. Since a different system is required, usually the skin tightening procedure is done at a later date after adipose tissue reduction treatment. In addition, even if an attempt is made to carry out the two treatments during a single visit of the patient to the clinic the patient or the equipment needs to be shifted from one location to another within the clinic, for example, to accommodate the need for a different system/technique to carry out the two different procedures.

Therefore, it would be desirable to provide a single step solution to the multiple step problem of either removing or reducing unwanted tissue volume while simultaneously providing for improved cosmetic appearance by skin contour smoothening.

### BRIEF DESCRIPTION

In one embodiment, a method for treating a region of interest using ultrasound is provided. The method comprises cavitating fat cells in the region of interest using one or more cavitating harmonics, and thermally treating connective tissues in the region of interest using one or more thermal harmonics.

In another embodiment, a method for treating a region of interest using ultrasound is provided. The method comprises cavitating fat cells in the region of interest using a cavitating pulse having one or more cavitating harmonics, and thermally treating connective tissues and adipose tissues in the region of interest using a thermal pulse having one or more thermal harmonics.

In yet another embodiment, a method for delivering a therapy using ultrasound is provided. The method comprises delivering ultrasound energy continuously at thermal harmonics to the region of interest, and periodically modulating the continuous ultrasound energy using a cavitating pulse having cavitating harmonics, wherein the thermal harmonics have a higher frequency than the cavitating harmonics.

In another embodiment, a system for providing non-invasive ultrasound based treatment to a region of interest is provided. The system comprises a transducer that generates a first set of cavitating harmonics, and a second set of thermal harmonics, and a control unit that controls the delivery of the first set of cavitating harmonics, and the second set of thermal harmonics, to the region of interest.

### DRAWINGS

These and other features, aspects, and advantages of the present invention will become better understood when the following detailed description is read with reference to the accompanying drawings in which like characters represent like parts throughout the drawings, wherein:

FIGS. 1-2 are schematic diagrams of two embodiments of an ultrasound device of the invention for treating fat cells and tightening the skin in a region of interest using ultrasound harmonics;

FIG. 3 is a block diagram of an ultrasound system of the invention; and

FIG. 4 is a flow chart for an example of a method of the invention for providing therapy to the region of interest.

### DETAILED DESCRIPTION

Embodiments of the invention relate to methods and systems for providing ultrasound based therapy. The therapy may be delivered non-invasively. The method of providing the therapy to a region of interest comprises treating fat cells in a region of interest using one or more harmonics; and tightening the skin in and/or around the region of interest using one or more other harmonics. In some embodiments, a single transducer is used for cavitating and thermally treating. In such embodiments, lower harmonics of a particular fundamental frequency may be used for cavitating fat cells in a region of interest; and the higher harmonics of the same fundamental frequency may be used for tightening the skin by thermally treating connective tissues. In other embodiments, separate transducers may be used for cavitating and thermally treating in a region of interest. In these embodiments where separate transducers are used for cavitating and thermally treating, the frequencies for the two treatments may be different. For example, for single element transducers, the transducer for cavitating may function at a fundamental frequency that is either higher or lower than a fundamental frequency of the transducer for thermally treating. However, the particular harmonics used for thermally treating will have higher frequency than the harmonics that is used for cavitating.

To more clearly and concisely describe the subject matter of the claimed invention, the following definitions are provided for specific terms, which are used in the following description and the appended claims. Throughout the specification, exemplification of specific terms should be considered as non-limiting examples.

As used herein, the term “harmonics” is a component frequency of the signal that is an integer multiple of the fundamental frequency. Harmonic frequencies are equally spaced by the width of the fundamental frequency and can be found by repeatedly adding the fundamental frequency. For example, if the fundamental frequency is 25 Hz, the frequencies of the harmonics are 25 Hz, 50 Hz, 75 Hz, 100 Hz.

As used herein, “cavitating” refers to treating tissue (such as fat tissue) primarily with cavitational mechanism. However, cavitating may also have thermal effects on the tissue. The tissue may be destroyed either by cavitation or thermal effect. HIFU parameters may be adjusted such that the majority of the damage to tissue is cavitational in nature, but there may be some thermal effects.

As used herein, “thermally treating” refers to treating tissue primarily with a mechanism that is thermal in nature. However, thermally treating may have cavitational effects.

As used herein, “adipose tissue” means subcutaneous, visceral or other tissues made primarily of fat cells. Adipose tissues may also comprise connective tissues, blood vessels and other structures. Adipose tissue may be white adipose tissue or brown adipose tissue.

As used herein, “connective tissues” are tissues that may be found either in the adipose tissue or in the skin surrounding the adipose tissue.

As used herein, “treating fat cells” refers to destroying fat cells by breaking, heating, or otherwise compromising the integrity, of the cellular membrane, causing the contents to flow out, often by viral, enzymic or osmotic mechanisms thereby compromising the integrity of the cells.

As used herein, “patient” means a person receiving the treatment for adipose tissue reduction and skin contour smoothening.

As used herein, “region of interest” means one or more sites associated with the patient targeted for receiving the therapy. The region of interest may or may not be imaged along with the therapy treatments. The region of interest may include, but is not limited to, an inner treatment region, a subcutaneous region of interest and/or any other region of interest in between the inner treatment region, and/or a subcutaneous region within a patient.

Terms “tightening the skin”, “skin tightening”, “skin contour smoothening”, “contour smoothening”, “contour irregularity smoothing” may be used interchangeably throughout the application, and are non-limiting.

A “therapy” as used herein may comprise a combination of treatments, for example, two different kinds of treatments such as, but not limited to, adipose tissue reduction, and skin contour smoothening, wherein the two procedures may be provided substantially contemporaneously. Therapy may also include cavitating and/or thermal treatment of diseased tissues, such as cancerous tissues.

As used herein, “substantially contemporaneously” means in a successive manner, without any compulsory time delays between the two treatments of adipose tissue reduction and skin tightening that are otherwise caused due to the requirement of the system or the method for treatment. For example, the therapy does not include any compulsory time delays, which are required when adipose tissue reduction, and skin contour smoothening employ different devices. The compulsory time delays in such cases may be to accommodate shifting of the patient from one device to another when switching from one treatment to another. The time delays may also include a subsequent appointment of the patient with the user, to receive the second treatment (skin contour smoothening) as it may not be feasible in the clinic settings to obtain the two different treatments from two different devices by one or more users on the same day. As used herein, “substantially contemporaneously” may also include simultaneously. However, cavitating and thermally treating may not be carried out in a successive manner. In some instances, the skin tightening (thermally treating) may be performed after a time delay after cavitating the adipose tissue for adipose tissue reduction, to allow the body to remove the treated adipose tissue (such as fat cells). In cases of multiple treatment sessions, the skin tightening may be performed on the second and subsequent treatments to tighten the skin in and around the regions where cavitating was carried out during the previous treatment session.

As used herein, “user” means one or more persons (technologist, nurse or physician) operating the system to provide therapy to the patient.

A “therapy session,” as used herein, is a period of time in which a patient receives therapy, e.g. for adipose tissue reduction and skin contour smoothening for a determined portion of the body. The entire therapy session for the determined portion of the body comprises of a single sitting, and the patient or the delivery device is not required to move around to accommodate the need for a different device to deliver the therapy. For example, a therapy session may include one visit by the patient to the user of the system. Therefore, the therapy sessions are time efficient and convenient for the recipient of the therapy, that is, the patient. However, a therapy session may include an extended period of time, where the extended period is required to cover a large treatment space of the body to deliver the therapy. The therapy session may also include any planned/intended time delays, or gaps between the therapy sessions, or between the two treatments, which are not because of the requirement of the system or the method for delivering the therapy.

In certain embodiments, a high intensity focused ultrasound (HIFU) transmitter provides two or more harmonics of high power ultrasound energy to a medical target region. The harmonics of the ultrasound signal have carefully chosen parameters for optimizing the effects of tissue damage. In one embodiment, one of the harmonics may modulate the other harmonics. For example, harmonics at higher frequencies may modulate harmonics at lower frequencies, regardless of the fundamental frequencies of the two harmonics. The lower frequency harmonics typically control growth of cavitation micro-bubbles in the focused region and are used for destroying the cells. Higher frequencies are used to thermally treat the target region. The thermal treatment may be provided before and/or after cavitating.

Harmonics used for cavitating may be variously referred to herein as “cavitating harmonics”. The cavitating harmonics may also provide thermal heating to the fat cells in addition to cavitating the fat cells. Harmonics used for thermally treating may be variously referred to herein as “thermal harmonics”. The thermal harmonics may sometimes cavitate the fat cells in addition to thermally treating the connective tissues. Generally, the cavitating harmonics have a lower frequency than the thermal harmonics. If the cavitating and the thermal harmonics share the same fundamental frequency, then the one or more cavitating harmonics are typically sub-harmonics of the one or more thermal harmonics. The frequency range for cavitating may be from about 100 KHz to about 2 MHz. The frequency range for thermally treating may be from about 2 MHz to about 10 MHz. In one or more of the examples of the methods, the cavitating harmonics and the thermal harmonics are high intensity focused ultrasound (HIFU).

Typically, different treatments require different frequencies of ultrasound energy. Different frequencies require different elements, as each element typically has a particular fundamental frequency. However, the invention offers a solution for providing the therapy including two or more treatments, using a single element transducer, thus making the therapy economical. In certain embodiments, use of harmonics of a particular fundamental frequency allows a single transducer to provide two or more kinds of treatments (such as cavitating and thermally treating). Depending on the range of frequency in which a particular harmonic falls, the harmonic may be applied for either treating fat cells or tightening the skin in a region of interest. Also, the single transducer element simplifies the electrical system design, and reduces the manufacturing cost of the system. For example, only a single transmitting channel may be required for transmission from the single element. The method is also applicable for ultrasound transducers that have two or more elements.

In some embodiments, the cavitating harmonics are delivered in a pulsed form, and the thermal harmonics are delivered continuously in ultrasound waveforms. In these embodiments, the continuous ultrasound energy may be modulated using the pulsed form.

In other embodiments, both the cavitating harmonics and the thermal harmonics are delivered in pulsed forms. One or more pulses of cavitating or thermal harmonics may be delivered substantially contemporaneously, simultaneously or sequentially. In one example, the thermal harmonics may be delivered prior to cavitating harmonics to increase the rate of treating the fat cells. Alternatively or in addition, the cavitating harmonics may be followed by thermal harmonics to thermally treat the region of interest after treating the fat cells.

In certain embodiments, the reduction of fat cells may be achieved by cavitating. Cavitating destroys the adipose cells by lysis. Subsequent thermal treatment of the surrounding connective tissues facilitates skin contour smoothening. According to some embodiments, the methods may be used to further treat surrounding tissue, such as muscle tissue, connective tissue, blood vessels, nerve tissue, fat tissue, adipose tissue and any combinations thereof.

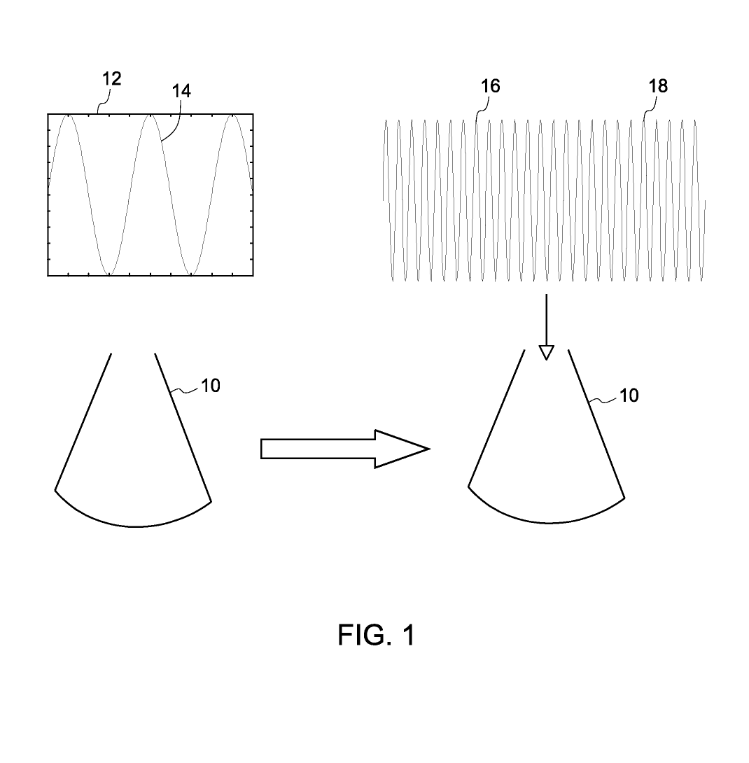

FIG. 1 illustrates a method for treating a region of interest using ultrasound. The method comprises treating the fat cells in the region of interest 10 using a cavitating pulse 12 having cavitating harmonics, represented by the reference numeral 14. After treating the fat cells, the region of interest 10 is thermally treated for tightening the skin using a thermal pulse 16 having thermal harmonics, represented by the reference numeral 18. The cavitating harmonics may have lower frequency than the thermal harmonics. For example, cavitating harmonics are sub-harmonics of the thermal harmonics. The cavitating harmonics in this example are in a range from about 100 KHz to about 1 MHz. The thermal harmonics are in a range from about 1 MHz to about 2.5 MHz.

In one embodiment, the duty cycle of the cavitating pulse and the duty cycle of the thermal pulse are substantially similar. In one example, the duty cycle of the cavitating pulse and the thermal pulse may be less than or equal to 30 percent. In one embodiment, the duty cycles of the cavitating pulse and the duty cycle of the thermal pulse may be different. In one example, the duty cycle of the cavitating pulse may be within 1 percent and the thermal harmonics may be a continuous wave. In one embodiment, where the duty cycle and the frequency of the cavitating pulse and the thermal pulse are substantially similar, the cavitational pulse may be achieved at power levels above the cavitation threshold and the thermal pulse is achieved at power levels below the cavitation threshold. In one example, where the duty cycle is about 5 percent, and the frequency is about 1 MHz, different power levels may be applied to have cavitational and thermal effects.

The process of cavitating the fat cells and thermally treating the connective tissues to tighten the skin may be iterative. That is, fat cells in a region of interest may be cavitated and/or the tightening of the skin may be performed more than once to obtain desirable results. For example, a cavitating pulse may be applied for a determined period of time, followed by a thermal pulse that may then be applied for another determined period of time. The time for applying the cavitating pulse may be same or different than the time for applying the thermal pulse. For example, the time for applying the cavitating pulse may depend on the amount of diseased tissue, or adipose tissue (for body contouring applications). The time for applying the thermal pulse may depend upon the amount of connective and skin tissues to be treated.

Applying both treating and thermal harmonics in pulse form enables lower temperature around the treated fat cells. In the case of both cavitating and thermal harmonics in pulse form, the acoustic power at the focal point of the ultrasound beam exceeds the cavitating threshold for a short duration of time and causes tissue cavitation.

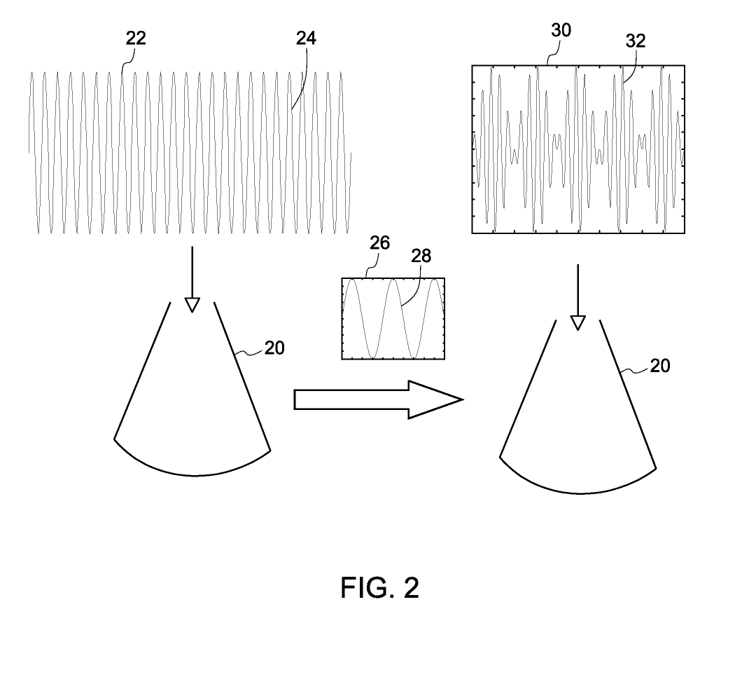

FIG. 2 illustrates a method for delivering therapy to a region of interest 20. The method comprises delivering the ultrasound wave 22 at thermal harmonics 24, and periodically modulating the ultrasound 22 using a cavitating pulse 26 having cavitating harmonics 28, wherein the thermal harmonics have a higher frequency than the cavitating harmonics. The acoustic power of the continuous wave 22 may be below a cavitating threshold. Depending on the tissue structure and tissue type, the thermal effect predominates when the acoustic pressure at the point of focus is below cavitation threshold of about 1 MPa to about 2 MPa. This thermal effect is due to the acoustic absorption of the tissue, which converts the mechanical energy of the acoustic wave into thermal energy. The cavitation effect becomes predominant when the acoustic power at the point of focus exceeds cavitation threshold.

Modulating the ultrasound wave 22 with the cavitating pulse 26 results in a modulated ultrasound wave 30. The modulation is determined by either adding or multiplying the frequency of the thermal harmonics and the cavitating harmonics. Multiplying the frequency allows for a larger difference in frequency while retaining the power. In the illustrated example, the frequency of the thermal harmonics and the cavitating harmonics is added. The thermal harmonics is in a range from about 1 MHz to about 30 MHz. The cavitating harmonics is in a range from about 100 KHz to about 3 MHz.

In one or more of the embodiments of the system, for providing non-invasive ultrasound based treatment to a region of interest, the system comprises a transducer that generates a first set of cavitating harmonics and a second set of thermal harmonics; and a control unit that controls the delivery of the first set of cavitating harmonics and the second set of thermal harmonics to the region of interest. In one example, providing the therapy may include cavitating the diseased tissues or fat cells in the region of interest.

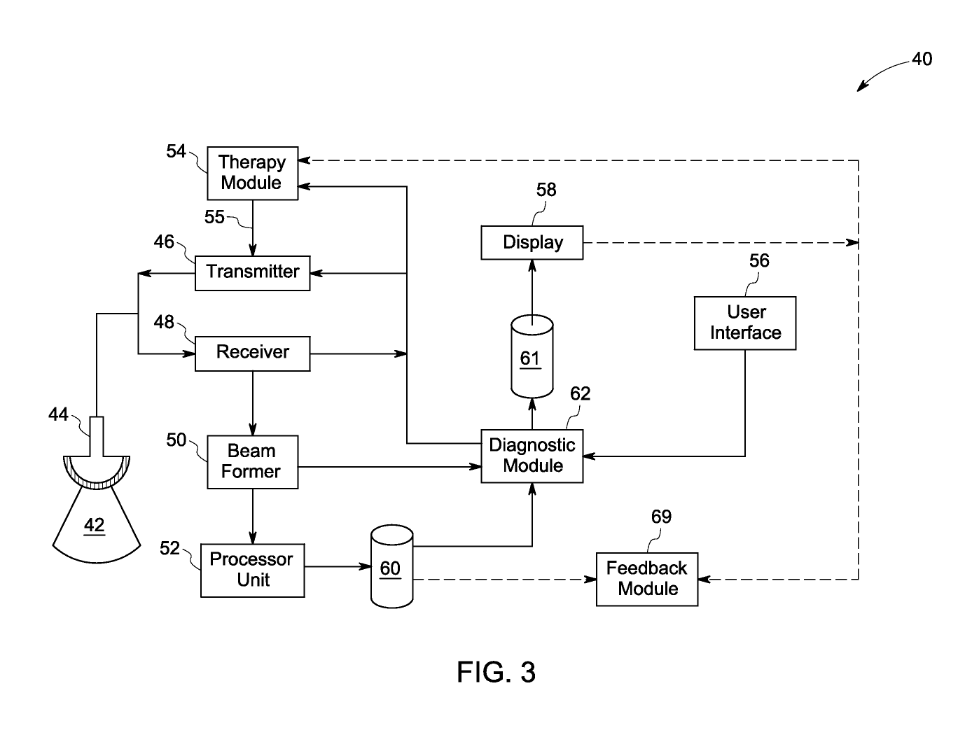

FIG. 3 illustrates an example of an ultrasound system 40 for providing therapy to the region of interest 42. In addition, the system 40 may image and display the images of the region of interest 42. The system 40 allows a user to substantially contemporaneously, simultaneously, or sequentially cavitate the fat cells and tighten the skin in a region of interest. In one example, the system 40 may be a console-based ultrasound system that may be provided on a movable base.

The system 40 comprises a probe housing 44 for housing at least one transducer. The transducer may employ a single element or an array of elements (two or more elements). The transducer provides therapy for the purpose of treating a region of interest that may be situated inside the body of a mammal, such as a human being. The transducer is preferably designed to deliver ultrasonic waves focused onto a focal point or a focal zone. The point of focal zone determines the tissue region that is to be treated. The transducer in the probe housing 44 may be used for cavitating the region of interest 42. Cavitating destroys the adipose tissue/cell by lysis. The transducer in the probe housing 44 may also provide thermal treatment either before or after cavitating. In the case of cavitating adipose tissue, subsequent thermal treatment of the surrounding connective tissues facilitates skin contour smoothening, for example. In the case of cavitating diseased tissues within the body of the patient, subsequent thermal treatment, facilitates at least partial joining of the tissues adjacent to the diseased tissues. According to some embodiments, the method may also affect surrounding tissues such as muscle tissue, connective tissue, blood vessels, nerve tissue, fat tissue, adipose tissue and any combinations thereof.

The ultrasound system 40 comprises a transmitter 46 that drives a single transducer element or an array of transducer elements within the probe 44 to direct ultrasonic signals into a body or volume. In one embodiment, where the probe 44 comprises a single transducer, also referred to as a therapy transducer, the transducer provides at least two harmonics, a first harmonics (of a particular fundamental frequency) for cavitating, and a second harmonics (of the same fundamental frequency) for thermally treating. The cavitating harmonics may be sub-harmonics of the thermal harmonics.

The system 10 may also include a separate imaging transducer (not shown). The imaging transducer may be integrated with the therapy transducer. The imaging transducer facilitates acquisition of image data. Further, the imaging transducer may be coupled to an imaging sub-system that may be configured to display an image representative of a region of interest 42 in the patient. The embodiments may be used in multi-modality systems that employ ultrasound therapy and imaging in conjunction with other imaging modalities or other sensor systems. The imaging transducer may image the region of interest 42 before applying the therapy, or after applying the therapy, or while applying the therapy. While only one region of interest 42 is depicted, the therapy transducer and the imaging transducer may operate in a plurality of regions of interest.

The therapy and imaging transducers comprise an array of transducer elements that emit ultrasonic signals. The transducer elements can comprise a piezoelectrically active material, such as lead zirconante titanate (PZT), lithium niobate, lead titanate, barium titanate, and/or lead metaniobate, or combinations thereof. Alternatively, the piezoelectrically active component of the transducer element may comprise one or more of a piezoelectric ceramic, a piezoelectric crystal, piezoelectric plastic, and/or piezoelectric composite materials. In addition to, or instead of, a piezoelectrically active material, transducers may comprise any other materials configured for generating radiation and/or acoustical energy such as capacitively coupled transducers or other acoustic sources. The transducers may also comprise one or more matching and/or backing layers configured along with the transduction element such as coupled to the piezoelectrically active material. In addition, the transducers may also be configured with single or multiple damping elements along the transducer element(s). A variety of geometries may be used and the probe 44 may be provided as part of, for example, different types of ultrasound probes.

The imaging transducer receives ultrasonic signals in response to ultrasonic signals transmitted by the therapy transducer for imaging purposes. In one example, the therapy transducer may deliver lower values of acoustic during imaging and relatively higher values of acoustic energy during therapy. The imaging transducer, in this example, operates in a frequency range from about 1 MHz to about 10 MHz. The transducer may operate in a frequency range from about 100 KHz to about 3 MHz, or from about 250 KHz to about 2 MHz, for cavitating the adipose tissues. The transducer may operate in a frequency range from about 1 MHz to about 30 MHz, or from about 1 MHz to about 10 MHz to thermally treat the region of interest 42.

The imaging signals are back-scattered from physiological structures in the body, for example, adipose tissue, muscular tissue, blood cells, veins or objects within the body (e.g., a needle, an implant) to produce echoes that return to the transducer element(s). The echoes are received by a receiver 48. The received echoes are provided to a beamformer 50 that performs beamforming and outputs an RF signal, for example. The RF signal is then provided to a processor unit 52 that processes the RF signal. Alternatively, the processor unit 52 may include a complex demodulator (not shown) that demodulates the RF signal to form IQ data pairs representative of the echo signals. The RF or IQ signal data may then be sent directly to a memory 60 for storage (e.g., temporary storage). Optionally, the output of the beamformer 50 may be passed directly to the diagnostic module 62.

The system 40 comprises therapy module 54 using the treatment device, such as the probe housing 44, to supply acoustic energy of two types, cavitating wave or pulse and thermal wave or pulse. The therapy module 54 is used to control the delivery of therapy to the treatment locations based on one or more therapy parameters. The therapy module 54 enables the transducer to produce one or more harmonics, such that some of these harmonics fall in the frequency range for cavitating, and the other harmonics fall in the frequency range for thermal treating. The cavitating pulse and the thermal pulse are delivered using a single transducer. The transducer generates high intensity focused ultrasound (HIFU). The cavitating harmonics are sub-harmonics of the thermal harmonics.

The therapy module 54 is connected to a user interface 56, such as a mouse, keyboard, and controls operation of the probe housing 44. The therapy module 54 is configured to receive inputs from a user via the user interface 56. In one embodiment, the therapy module 54 may automatically move the treatment location between multiple points based on user inputs. The therapy module 54 of the system 40 may also be used to enable the user to selectively control the delivering of cavitating and thermal harmonics.

The therapy module 54 may be configured to receive therapy commands (and imaging commands if the region of interest 42 is being imaged) from the user. The therapy module 54 may receive imaging and/or therapy commands from the user through a user interface 56 for applying therapy to the region of interest 42. The user may provide instructions on whether to image the region of interest 42, or provide therapy to the region of interest 42. Further, the user can specify whether to cavitate the fat cells or perform skin tightening for the region of interest 42. The delivery of therapy may be based upon therapy commands provided by the user. The user interface 56 may be a touchscreen, keyboard, or a mouse. For example, the touchscreen may allow the operator or user to select options by touching displayed graphics, icons, and the like.

A therapy command may comprise any factor or value that may be determined by the system 40 or any input that may be entered by the user that affects the therapy applied to the region of interest 42. In some embodiments, the system 40 may automatically differentiate the adipose tissue and the non-adipose tissue (such as connective tissue). The system 40 may also automatically display to a viewer (such as the user or the patient) a boundary between the targeted tissue (e.g., a diseased tissue or adipose tissue) and the non-targeted tissue (e.g., surrounding healthy tissue, or non-adipose tissue) by overlaying the image with a graphical representation that indicates the boundary. Furthermore, the system 40 may automatically display to a viewer of the system 40 the region of interest 42 within the image where therapy may be applied (or is recommended by the system 40 to be applied). In addition, the user may be able to modify the treatment space 40 that is automatically displayed by the system 40 through user inputs.

Examples of the therapy command may comprise parameters of the ultrasound transducer, or time period for applying the therapy. For example, the therapy command may comprise a transducer parameter that relates to the configuration or operation of the transducer elements (not shown) or probe housing 44. The terms “therapy commands” and “therapy parameters” may be used interchangeably, and refer, for example, to the settings of the system or the factors regarding the patient that are taken into account for delivering the therapy. In one embodiment, the therapy command may include instructing the system 40 to deliver low energy pulses during imaging and high-energy pulses during therapy.

Examples of a transducer parameter include, but are not limited to, a focal depth of the ultrasound beam, a focal region size, an ablation time for each point within the region of interest that receives therapy, an energy level of the therapy signals, and a rate of focal region movement within the ROI during the therapy session. The transducer parameters may also include a frequency or intensity of the therapy ultrasound signals, power, peak rarefactional pressure, pulse repetition frequency and length, duty cycle, depth of field, waveform used, speed of beam movement, density of beam, cavitation priming pulse, and general pulse sequence parameters. Also, therapy commands may include anatomical parameters, such as the location, shape, thickness, and orientation of adipose tissue and non-adipose tissues. An anatomical parameter may also include the density of the adipose tissue and the non-adipose tissues. Furthermore, therapy parameters may include the type of probe 64 used during the therapy session. The age, gender, weight, ethnicity, genetics, or medical history of the patient may also be examples of therapy commands. After therapy has been applied to a region of interest 42, the system 40 or the operator/user may adjust the therapy parameters before applying therapy to the same region of interest 42 again, or to another region of interest.

The therapy module 54 may be implemented utilizing any combination of dedicated hardware boards, DSPs, processors, etc. Alternatively, the therapy module 54 may be implemented utilizing an off-the-shelf PC with a single processor or multiple processors, with the functional operations distributed between the processors. As a further option, the therapy module 54 may comprise a hybrid configuration in which certain modular functions are performed utilizing dedicated hardware, while the remaining modular functions are performed utilizing an off-the-shelf PC and the like.

While the therapy module 54 is configured to deliver therapy to the treatment locations based on one or more therapy parameters, the diagnostic module 62 is configured to control the probe 44 to obtain diagnostic ultrasound signals from the region of interest 42. The processor unit 66 processes the acquired ultrasound information (e.g., RF signal data or IQ data pairs) and prepares frames of ultrasound information for display on a display 58. The display 58 may comprise one or more monitors that present patient information, such as diagnostic and therapeutic ultrasound images, to the user 44 for review, diagnosis, analysis, and/or treatment. The display 58 may automatically display, for example, a (two dimensional) 2D, (three dimensional) 3D, or (four dimensional) 4D ultrasound data set stored in the memory 60 or currently being acquired, this stored data set may also be displayed with a graphical representation (e.g., an outline of a treatment space or a marker within the treatment space).

The processing unit 66 may receive ultrasound data in several forms. For example, in the embodiment, the received ultrasound data constitutes IQ data pairs representing the real and imaginary components associated with each data sample. The data may be processed by the processing unit 66 by employing one or more of a color-flow module, an acoustic radiation force imaging (ARFI) module, a B-mode module, a spectral Doppler module, an acoustic streaming module, a tissue Doppler module, a C-scan module, and an elastography module. Other modules may be included, such as an M-mode module, power Doppler module, harmonic tissue strain imaging, among others. However, embodiments are not limited to processing IQ data pairs. For example, processing may be done with RF data and/or using other methods. Furthermore, data may be processed through multiple modules.

Each of the modules are configured to process the IQ data pairs in a corresponding manner to generate color-flow data, ARFI data, B-mode data, spectral Doppler data, acoustic streaming data, tissue Doppler data, C-scan data, elastography data, among others, all of which may be stored in a memory 60 temporarily before subsequent processing. As an example, the system may be configured to view different ultrasound images relating to a therapy session in real-time on the display 58.

The processing unit 66 is adapted to perform one or more processing operations according to a plurality of selectable ultrasound modalities on the acquired ultrasound information. Acquired ultrasound information may be processed in real-time during a scanning or therapy session as the echo signals are received. Additionally or alternatively, the ultrasound information may be stored temporarily in the memory 60 during a scanning session and processed in less than real-time in a live or off-line operation. For example, the image memory 61 may be used to store processed frames of acquired ultrasound information that are not scheduled to be displayed immediately. The image memory 61 may comprise any known data storage medium, for example, a permanent storage medium and/or removable storage mediums.

After or while providing therapy to an area within the region of interest 42, the user may determine, whether the therapy is complete for the region of interest 42 and whether the region of interest should be moved to another point within the patient. Automatic determination of whether the treatment space has been sufficiently treated or completed may be determined by, for example, elasto-graphic methods. The user or a feedback module 69 may also determine whether the therapy is complete. If the therapy is complete for a given region of interest, the feedback module 69 may determine the next region of interest where the probe housing 44 should be moved.

The feedback module 69 may be coupled to the processing unit 66. In addition, the feedback module 69 may also be coupled to one or more of the display 58, memory 60, image memory 61, or the user interface 56. The feedback module 69 may compare the actual output of the system with the desired output. The actual output refers to the result of the therapy delivered to the region of interest. The actual output may be provided as displayed images, or images stored in the memory 60 or 61, or the data related to the displayed or stored images. The output may be in a tabular form, or images, that may be stored in the memory 60 or 61. The output may also be specified by the user, for example, depending on the amount of adipose tissue to be reduced.

The feedback module 69 may compare the actual output and the desired output and inform/alert the system if required. In one example, the feedback module 69 may also use the therapy commands provided to the system 40 by the user to determine the acceptable levels of adipose tissue reduction and thermal treatment, and notify the system when such limits are exceeded or approaching. In one example, the feedback module 69 may alert the system by beeping, to caution the user, for example, if the determined limit of fat cells to be treated exceeds or is about to exceed that limit In one embodiment, the feedback module 69 may have built in intelligence that may alter the therapy parameters to amend the therapy being provided.

The feedback module 69 may take the data in the processing unit 66, displayed images (on the display 58), or stored images (in the memory 61) as the input and make a decision whether the data or the images are acceptable. For example, the feedback module 69 may use the displayed images to determine whether the amount of fat cells cavitated in the region of interest 42 is sufficient to stop the therapy in the region of interest 42. The feedback module 69 may either provide feedback after completion of the therapy or during the therapy. In one example, the feedback module 69 may verify whether the amount of cavitated fat cells in a given portion is acceptable by comparing the actual amount of the cavitated fat cells with the value of the cavitated fat cells calculated using the therapy parameters. If, for example, the depth of the fat cells that are cavitated exceeds, or is about to exceed a determined value, the feedback module 69 may be configured to raise an alarm, such as a continuous beep, till the user acknowledges receiving the beep (e.g. by means of the user interface 56). The user may then review the information from the feedback module 69. In this manner, the feedback module 69 may prevent any inadvertent errors that could otherwise happen due to human error.

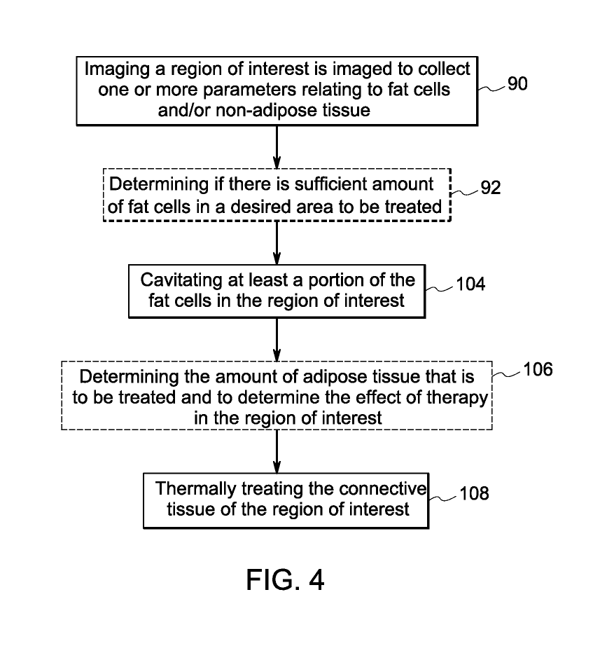

FIG. 4 is a flow chart for a non-invasive method for substantially contemporaneously cavitating and thermally treating a region of interest. At block 90, a region of interest is imaged to collect one or more parameters relating to fat cells and/or non-adipose tissue (such as connective tissue). The region may be imaged either before and/or after delivering the therapy.

The imaging may be completed before delivering the therapy, for example, to collect the parameters such as but not limited to, anatomical parameters, such as the location, shape, thickness, and orientation of adipose tissue and non-adipose tissues. The delivery of the therapy may be planned according to the parameters determined using imaging, and any other factors already known to the user, or provided by the patient. An anatomical parameter may also include the density of the fat cells and the non-adipose tissues.

Optionally, at block 92, before delivering the therapy, the user may use an imaging instrument such as a diagnostic ultrasound device, an MRI device, a X-ray device, or a DXA (Dual energy x-ray attenuation) device to determine if there is sufficient amount of fat cells in a desired area to be treated using HIFU energy. Alternatively, determining a volume of fat cells to be treated may include tests such as a manual pinch test or caliper test carried out by a physician to determine if a patient has sufficient fat cells at a particular site to warrant the procedure. The safety measure and standard used by such a test may also satisfy the minimum requirements of a HIFU procedure.

Once the volume of tissue is identified, the user may determine the corresponding surface area over the volume that can be treated. The user may create one or more contour lines as part of the treatment-planning phase prior to commencing the therapy. During this step the physician may draw or otherwise indicate on a patient skin surface, a region that can safely be treated using a HIFU transducer.

While the depth of the fat cells should be sufficient to allow the focal zone of the HIFU transducer to be safely focused in the fat cells with some margin of safety both above and below the focal point of the transducer. Varying the focal depth of the transducer, as well as the shape and focus of the transducer allows for more precise control over the delivery of ultrasound (HIFU) energy, while simultaneously reducing the clearance zones needed for safe operation.

Once the pre-treatment steps of determining a volume of adipose tissue or fat cells to be treated, and identifying and/or making a corresponding surface area of skin over the volume of fat cells are completed, an ultrasound probe is moved over the identified region to ablate the underlying fat cells, and to smooth and tighten the skin. There are a few major factors affecting the frequency selection. For example, the depth of the treatment zone may determine the choice of ultrasound frequency. The choice of a lower frequency to penetrate to deeper tissue may negatively impact the energy deposition, as the lower frequencies are not absorbed at the same rate as higher frequencies. Also, the size of the focus spot may determine the ultrasound frequency. The ultrasound beam with higher frequency may be focused in a smaller region for better spatial resolution. A small focus spot is useful for small treatment areas and thin tissue regions. Small focus region usually means low coverage and slow speed. During a therapy session, the ultrasound frequency may vary from one region of interest to another. For example, a higher frequency may be more appropriate for tissue ablation at small, curvy areas.

At block 104, at least a portion of the fat cells in the region of interest is treated, such as cavitated, using acoustic energy delivered by the transducer. The harmonics for cavitating the fat cells may have a frequency in a range from about 100 KHz to about 3 MHz, or from about 250 KHz to about 2 MHz. The selection of harmonics for treating may depend upon power of the transducer, duty cycle of the transducer, and the like. The selection of the frequency may depend on several factors, such as but not limited to, treatment zone, focus spot, or the region of interest. In one example, use of a lower frequency to penetrate to a tissue disposed deep inside the patient may negatively impact the energy deposition as the lower frequencies may not be absorbed at the same rate as higher frequencies. In another example, ultrasound beam may be focused in a smaller focal spot with higher frequency for better special resolution. Smaller focal spots are advantageous for small treatment areas and thin tissue region. However, small focus region usually translates to low coverage and slow speed. During a therapy session, the frequency might vary from one region of interest to another. For example, a higher frequency might be more appropriate for treating small, and curvy areas.

The transducer is moved over the surface area. The transducer emits energy to the focal zone in sufficient strength (power) and intensity (pressure) for cavitating the fat cells. If the transducer is moved in a continuous manner such that a single linear lesion field is formed along the path or axis of motion, the lesion field is said to be contiguous, or a contiguous lesion field. A volume of over lapping lesion field produced from more than one scan line (such as an intersection) forms a cooperative lesion field.

In one embodiment, the HIFU energy may be applied in a manner to form a pattern of discrete ablated field and non-ablated fields around the ablated fields within a region of interest. In another embodiment, the HIFU may be applied in a manner that divides the region of interest into a plurality of smaller treatment sites, and the sum of the treatment sites produces the desired coverage to form the region of interest. HIFU energy may be applied in either continuous or discontinuous motion through individual treatment sites. The various treatment sites, which form the region of interest, may be uniform or different in size.

Optionally, at block 106, imaging may be carried out to determine the amount of adipose tissue that is to be treated and to determine the effect of therapy in the region of interest. Depending on the amount of the cavitated fat cells, a decision may be taken by the user or the system whether to continue treating, or to switch to thermally treating to tighten the skin. Also, a decision may be made by the user or the system whether the therapy needs to be continued in the same region of interest, or if a new region of interest needs to be selected.

At block 108, the connective tissue of the region of interest, for example, the connective tissue surrounding the treated fat cells are thermally treated by thermal harmonics. The frequency range of the thermal harmonics may be in a range from about 1 MHz to about 30 MHz, or from about 1 MHZ to about 10 MHz. In one embodiment, the thermal treatment immediately follows the cavitation. An imaging step may also be performed at any point before, after, between or during the steps of cavitating the fat cells and tightening the skin. In certain embodiments, imaging may be carried out after tightening the skin by thermally treating the connective tissues to determine whether to continue the treatment of the determined region of interest or move on to the next region of interest in the subject.

Imaging may be carried out in real time, or an image of the region of interest may be created after every treatment step. In one example, the image may be a B-mode, or an elastography image. The user or the feedback module may refer to the images acquired to verify that the treatment delivered to the region of interest is producing desired results or the treatment needs to be modified. For example, the user or the feedback module may refer to the images to confirm if the delivery of the calculated treatment dose produced the desired results, or if more treatment is required to cavitate the adipose layer to the desired thickness. Also, the decision to discontinue/stop the therapy, to a particular region of interest, may be based on the images acquired of the region of interest before, during or after the therapy.

As noted, the steps of imaging, cavitating and thermally treating may be accomplished by delivering the corresponding acoustic energy to the region of interest. The acoustic energy may be delivered using two or more ultrasound transducers disposed in a single housing. As noted, the imaging transducer may be integrated with the therapy transducer. In one embodiment, the therapy transducer comprises a plurality of elements. Some of the elements may be used for cavitating fat cells, and the other elements may be used for tightening the skin in and around the region of interest. In another embodiment, the therapy transducer may have a single element that produces two or more harmonics. One of the harmonics may be used for treating the fat cells and the other for tightening the skin in and around the region of interest. The parameter of the transducer may be altered to switch between the step of cavitating and thermally treating by switching from one harmonic to another. If a single transducer is being used for both cavitating and thermally treating, the transducer may switch between the step of cavitating and thermally treating by altering one or more parameters such as, but not limited to, duty cycle, power, speed, time of treatment, frequency, or combinations thereof. The steps of cavitating and thermally treating may be applied in the same therapy session, and using the same device. In one embodiment, the step of thermally treating may be applied before cavitating.

The combination of cavitating and thermally treating may be applied in several different modes. In one example, the step of cavitating the fat cells comprises cavitating the fat cells over the entire region of interest, and the step of thermally treating refers to applying thermal treatment subsequent to the step of cavitating the fat cells and comprises thermally treating the connective tissue over the entire region of interest. In another example, cavitating the fat cells at a location within the region of interest may be followed by tightening the skin at that location before shifting the probe to the next location within the region of interest.

When therapy is applied, ultrasonic therapy energy (e.g., HIFU) from the probe is directed toward a region of interest. A treatment location within the region of interest may be defined as a region where a therapy beam formed by ultrasound signals from the transducer elements is focused. For instance, the treatment location may be defined as the focal region of the transducer elements. The therapy beam may be shaped and directed by a selected configuration and operation of the transducer elements. As such, the treatment location may vary in size and shape within a single therapy session.

The therapy location throughout the region of interest may be moved between multiple points or locations. As used herein, “moving the treatment location between multiple points” includes, but is not limited to, moving the treatment location along a therapy path between a first point and an end point, and also may comprise moving the treatment location to separate and distinct points within the region of interest that may or may not be adjacent to one another along a path. The therapy path may take the form of separate points where therapy is applied. For example, therapy may first be applied to a first point. After therapy has been applied to the first point, the focal region may be readjusted onto a second point along the therapy path that is separate and remotely spaced from the first point. Therapy may then be applied to the second point. The process may continue along the therapy path until the therapy session is concluded at an end point. In other embodiments, the therapy may be continuously applied as the focal region is moved along the therapy path in a sweeping manner. For example, therapy may be continuously applied as the treatment location is moved between the first point and the end point.

Parameters of the HIFU transducer may be adjusted to produce the desired treatment needed to destroy adipose tissue and denature collagen fibrils. Moving the HIFU transducer and applying therapeutic ultrasound energy generally do not produce lesion or halo fields that extend beyond the dimensions of the adipose tissue volume.

The embodiments described above may be used to treat various kinds of tissues within the body. For example, the above-described embodiments may be used to image and treat a tumor within a region of interest. With respect to diseased tissue, various embodiments may be used to automatically identify the tumor and/or to allow user inputs to identify treatment spaces within a region of interest and to set therapy parameters for the treatment. Furthermore, one or more of the embodiments may be used for palliative treatments for cancer, thermal treatment of muscles, or ultrasonically activating drugs, proteins, stem cells, vaccines, DNA, and gene delivery.

While only certain features of the invention have been illustrated and described herein, many modifications and changes will occur to those skilled in the art. It is, therefore, to be understood that the appended claims are intended to cover all such modifications and changes as fall within the scope of the invention.

1. A method for treating a region of interest using ultrasound, comprising:
cavitating fat cells in the region of interest using one or more cavitating harmonics; and
thermally treating connective tissues in the region of interest using one or more thermal harmonics.

2. The method of claim 1, wherein the one or more cavitating harmonics are sub-harmonics of the one or more thermal harmonics.

3. The method of claim 1, wherein the cavitating harmonics are delivered in a pulsed form, and wherein the thermal harmonics are delivered in a continuous wave form.

4. The method of claim 1, wherein both the cavitating harmonics and the thermal harmonics are delivered in pulsed forms.

5. A method for treating a region of interest using ultrasound, comprising:
cavitating fat cells in the region of interest using a cavitating pulse having one or more cavitating harmonics; and
thermally treating connective tissues and adipose tissues in the region of interest using a thermal pulse having one or more thermal harmonics.

6. The method of claim 5, wherein the cavitating and thermal pulses are applied alternatingly.

7. The method of claim 5, wherein the cavitating and thermal pulses are applied simultaneously.

8. The method of claim 5, wherein the cavitating harmonics are sub-harmonics of the thermal harmonics.

9. The method of claim 5, wherein the cavitating harmonics are in a range from about 100 KHz to about 3 MHz.

10. The method of claim 5, wherein the thermal harmonics are in a range from about 1 MHz to about 30 MHz.

11. The method of claim 5, wherein the cavitating pulse and the thermal pulse are high intensity focused ultrasound (HIFU).

12. The method of claim 5, wherein a duty cycle of the cavitating pulse and a duty cycle of the thermal pulse are substantially similar.

13. The method of claim 12, wherein a frequency of the cavitating pulse and the thermal pulse are substantially similar, and wherein a power level for the cavitating pulse is higher than a power level for the thermal pulse.

14. The method of claim 5, wherein the cavitating pulse and the thermal pulse are delivered using a single transducer.

15. The method of claim 14, wherein the single transducer comprises a single element.

16. A method for delivering a therapy using ultrasound to a region of interest, comprising:
delivering ultrasound energy continuously at thermal harmonics to the region of interest; and
periodically modulating the continuous ultrasound energy using a cavitating pulse having cavitating harmonics, wherein the thermal harmonics have a higher frequency than the cavitating harmonics.

17. The method of claim 16, wherein the modulation is provided by multiplying the frequency of the cavitating harmonics and the thermal harmonics.

18. The method of claim 16, wherein an acoustic power of the continuous ultrasound energy is below a cavitating threshold.

19. The method of claim 16, wherein the thermal harmonics is in a range from about 1 MHz to about 30 MHz.

20. The method of claim 16, wherein the cavitating harmonics is in a range from about 100 KHz to about 3 MHz.

21. A system for providing non-invasive ultrasound based treatment to a region of interest, the system comprising:
a transducer that generates a first set of cavitating harmonics, and a second set of thermal harmonics; and
a control unit that controls the delivery of the first set of cavitating harmonics, and the second set of thermal harmonics, to the region of interest.

22. The system of claim 21, wherein the transducer comprises a single element.

23. The system of claim 21, wherein the transducer generates high intensity focused ultrasound (HIFU).

24. The system of claim 21, wherein the first set of harmonics are sub-harmonics of the second set of harmonics.
